(19) United States
(12) Patent Application Publication
(10) Pub. No.: US 2016/0316821 A1
(43) Pub. Date: Nov. 3, 2016

(54) ELECTRONIC CIGARETTE WITH LIMITED SERVICE LIFE AND METHOD FOR LIMITING SERVICE LIFE OF ELECTRONIC CIGARETTE

(71) Applicant: HUIZHOU KIMREE TECHNOLOGY CO., LTD. SHENZHEN BRANCH, Shenzhen (CN)

(72) Inventor: Qiuming LIU, Huizhou (CN)

(73) Assignee: HUIZHOU KIMREE TECHNOLOGY CO., LTD. SHENZHEN BRANCH, Shenzhen (CN)

(21) Appl. No.: 15/206,566

(22) Filed: Jul. 11, 2016

Related U.S. Application Data
(63) Continuation of application No. PCT/CN2014/070977, filed on Jan. 21, 2014.

(30) Foreign Application Priority Data
Jan. 13, 2014 (CN) 201410014201.7

(51) Int. Cl. A24F 47/00; A61M 11/04; A61M 15/06

(52) U.S. Cl. CPC A24F 47/008; A61M 15/06; A61M 11/042; A61M 2016/0018

(57) ABSTRACT

An electronic cigarette with a limited service life includes a battery, a switch assembly and an atomizer, and further includes a control module and a smoking trigger module. The control module includes a counting sub-module and a main control sub-module; the counting sub-module is connected to the main control sub-module, and is configured to record the number of puffs according to a monitoring signal transmitted from the smoking trigger module to the main control sub-module; and the main control sub-module is connected to the battery, the switch assembly and the smoking trigger module, and is configured to control the electronic cigarette according to the number of puffs and the monitoring signal, and permanently disable the electronic cigarette in a case that the number of puffs reaches a preset value.

### CROSS REFERENCE OF RELATED APPLICATION

This application is a continuation of International Application No. PCT/CN2014/070977, filed on Jan. 21, 2014, which claims the benefit of priority to Chinese Patent Application No. 201410014201.7, titled “ELECTRONIC CIGARETTE WITH LIMITED SERVICE LIFE AND METHOD FOR LIMITING SERVICE LIFE OF ELECTRONIC CIGARETTE”, filed with the Chinese State Intellectual Property Office on Jan. 13, 2014, both of which are hereby incorporated by reference in their entireties.

### FIELD

The present application relates to the technical field of electronic cigarettes, and in particular to an electronic cigarette with limited service life and a method for limiting a service life of an electronic cigarette.

### BACKGROUND

An electronic cigarette is a common electronic product simulating a cigarette, and mainly consists of a battery rod and an atomizer. When a smoker inhales, the atomizer is turned on by a switch assembly electrically connected to the atomizer. After the atomizer is turned on, a heat-generating wire generates heat to evaporate and atomize e-liquid, thereby forming a vapor simulating a cigarette smoke.

For a conventional disposable electronic cigarette, the taste of the vapor is pure during an earlier period of its service life since the electricity quantity of a battery and the quantity of the e-liquid are sufficient. However, during a later period of its service life, due to the insufficient electricity quantity or the insufficient quantity of the e-liquid or the like, the amount of the vapor is reduced, and a smell may even be caused by burning cotton, and in this case, the disposable electronic cigarette does not remind the user, which brings a bad experience to the user.

Therefore, in view of the above situation, an important technical issue to be addressed presently by the person skilled in the art is to improve the structure of the electronic cigarette, to allow the electronic cigarette to be more human-friendly and bring a good experience to the user.

### SUMMARY

An electronic cigarette and a method for limiting a service life of an electronic cigarette are provided according to embodiments of the present application. A counter for counting the number of puffs and a detector for detecting an electricity quantity of a battery are provided, and in the case that the number of puffs reaches a preset value or the electricity quantity is low, the operation of the electronic cigarette is directly stopped or is stopped after a warning is given, to permanently disable the electronic cigarette, thereby ensuring a product consistency and bringing a good smoking experience to the user.

An electronic cigarette with a limited service life according to an embodiment of the present application includes a battery, a control module, a switch assembly, an atomizer and a smoking trigger module. The control module includes a counting sub-module and a main control sub-module; the counting sub-module is connected to the main control sub-module, and is configured to record the number of puffs according to a monitoring signal transmitted from the smoking trigger module to the main control sub-module. The main control sub-module is connected to the battery, the switch assembly and the smoking trigger module, and is configured to control the electronic cigarette according to the number of puffs and the monitoring signal, and permanently disable the electronic cigarette in a case that the number of puffs reaches a preset value.

Optionally, the smoking trigger module may include an airflow sensor and/or a button input module.

Optionally, the smoking trigger module may be the airflow sensor and is installed inside an airflow channel of the electronic cigarette.

Optionally, the control module may further include an anti-interference sub-module; and the anti-interference sub-module is configured to record a trigger hold time of the airflow sensor, and transmit a signal indicating that a present record is invalid to the main control sub-module in a case that the trigger hold time is less than a preset value.

Optionally, the switch assembly may be a transistor switch, and the transistor switch is electronically connected to the battery, the main control sub-module and the atomizer.

Optionally, the control module may further include an electricity quantity detection sub-module; and the electricity quantity detection sub-module is connected to the main control sub-module and is configured to detect an electricity quantity of the battery.

Optionally, the electronic cigarette may further include a warning module; wherein the warning module is connected to the main control sub-module, and is configured to give a warning in response to a control signal of the main control sub-module in a case that the number of puffs counted by the counting sub-module is about to reach or reaches the preset value.

A method for limiting a service life of an electronic cigarette according to the embodiments of the present application is provided to limit the service life of the above electronic cigarette. The method includes:
S1, receiving a monitoring signal of the smoking trigger module;
S2, recording the number of puffs according to the monitoring signal; and
S3, controlling the electronic cigarette according to the number of puffs and the monitoring signal, and permanently disabling the electronic cigarette in a case that the number of puffs reaches a preset value.

Optionally, before the step S3, the method may further includes:
detecting an electricity quantity of the battery of the electronic cigarette.

Optionally, in the method,
the step S1 may further include receiving, by the main control sub-module, the monitoring signal of the smoking trigger module;
the step S2 may further include recording, by the counting sub-module, the number of puffs according to the monitoring signal; and
the step S3 may further include controlling, by the main control sub-module, the electronic cigarette according to the number of puffs and the monitoring signal, and controlling the battery to stop supplying power to the atomizer in the case that the number of puffs reaches the preset value, to permanently disable the electronic cigarette.

The electronic cigarette with a limited service life according to the present application includes the battery, the control module, the switch assembly, the atomizer and the smoking trigger module. The control module includes the counting sub-module and the main control sub-module. The counting sub-module is connected to the main control sub-module and is configured to record the number of puffs according to the monitoring signal transmitted from the smoking trigger module to the main control sub-module. The main control sub-module is connected to the battery, the switch assembly and the smoking trigger module, and is configured to control the electronic cigarette according to the number of puffs and the monitoring signal. With the counter for counting the number of puffs and the detector for detecting an electricity quantity of the battery, the electronic cigarette with a limited service life and the method for limiting the service life of an electronic cigarette according to the present application can directly stop the operation of the electronic cigarette or stop the operation of the electronic cigarette after giving a warning in the case that the number of puffs reaches a preset value or the electricity quantity is low, to permanently disable the electronic cigarette, thereby ensuring a produce consistency and bringing a good smoking experience to a user.

### BRIEF DESCRIPTION OF THE DRAWINGS

For more clearly illustrating embodiments of the present application or the technical solutions in the conventional technology, drawings referred to describe the embodiments or the conventional technology will be briefly described hereinafter. Apparently, the drawings in the following description are only some embodiments of the present application, and for the person skilled in the art, other drawings may be obtained based on these drawings without any creative efforts.

FIG. 1 is a schematic view showing the structure of a first embodiment of an electronic cigarette with a limited service life according to the present application;

FIG. 2 is a schematic view showing the structure of a second embodiment of the electronic cigarette with a limited service life according to the present application;

FIG. 3 is a schematic view showing the structure of a third embodiment of the electronic cigarette with a limited service life according to the present application;

FIG. 4 is a schematic view showing the structure of a fourth embodiment of the electronic cigarette with a limited service life according to the present application;

FIG. 5 is a schematic view showing the structure of a fifth embodiment of the electronic cigarette with a limited service life according to the present application;

FIG. 6 is a schematic circuit diagram of an embodiment of an electronic cigarette with a limited service life according to the present application;

FIG. 7 is a flowchart of a first embodiment of a method for limiting a service life of an electronic cigarette according to the present application; and

FIG. 8 is a flowchart of a second embodiment of a method for limiting a service life of the electronic cigarette according to the present application.

### DETAILED DESCRIPTION OF EMBODIMENTS

An electronic cigarette with a limited service life and a method for limiting a service life of an electronic cigarette are provided according to embodiments of the present application, in which a counter for counting the number of puffs and a detector for detecting an electricity quantity of a battery are provided, and in the case that the number of puffs reaches a preset value or the electricity quantity is low, the operation of the electronic cigarette is directly stopped or is stopped after a warning is given, to permanently disable the electronic cigarette, thereby ensuring a product consistency and bringing a good smoking experience to the user.

The technical solutions according to embodiments of the present application are described clearly and in detail hereinafter in conjunction with the drawings in the embodiments of the present application. Apparently, the embodiments described are only a part of the embodiments of the present application, rather than all embodiments. Based on the embodiments in the present application, all of other embodiments, made by the person skilled in the art without any creative efforts, fall into the scope of the present application.

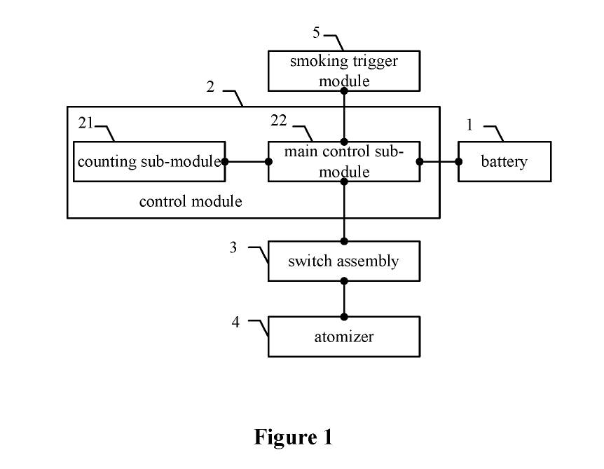

Referring to FIG. 1, a first embodiment of an electronic cigarette with a limited service life according to the present application includes a battery 1, a control module 2, a switch assembly 3, an atomizer 4 and a smoking trigger module 5. The control module 2 includes a counting sub-module 21 and a main control sub-module 22. The counting sub-module 21 is connected to the main control sub-module 22, and is configured to record the number of puffs according to a monitoring signal transmitted from the smoking trigger module 5 to the main control sub-module 22. The main control sub-module 22 is connected to the battery 1, the switch assembly 3 and the smoking trigger module 5, and is configured to control the electronic cigarette according to the number of puffs and the monitoring signal.

Optionally, the smoking trigger module 5 includes an airflow sensor and/or a button input module.

The above control module 2 may be embodied as a single chip computer, and the counting sub-module 21 and the main control sub-module 22 of the control module 2 may be respectively a counter and a controller of the single chip computer. When a signal transmitted by the smoking trigger module 5, such as a rising edge or a falling edge of the signal, is detected by the controller via a pin of the single chip computer, the controller transmits a counting signal to the counter, and the counter records the number of puffs. Of course, the control module 2 may be implemented by other microprocessors or programmable logical devices.

The electronic cigarette with a limited service life according to the present application includes the battery 1, the control module 2, the switch assembly 3, the atomizer 4 and the smoking trigger module 5. The smoking trigger module 5 includes the airflow sensor and/or the button input module. When a smoker inhales, the airflow sensor senses an airflow and then generates a monitoring signal, or the smoker presses the button input module to generate a monitoring signal. The main control sub-module 22 generates a switch instruction according to the detected monitoring signal, transmits the switch instruction to the switch assembly 3, and turns on the atomizer 4 via the switch assembly 3. After the atomizer 4 is turned on, a heat-generating wire generates heat to evaporate and atomize the e-liquid, to form a vapor simulating cigarette smoke. At the same time, this smoking action triggers the smoking trigger module 5, and the counting sub-module 21 records the number of puffs. When the number of puffs reaches a preset value, the main control sub-module 22 controls the switch assembly 3 to be turned off, and controls the battery 1 to stop supplying power to the atomizer 4, thereby stopping the operation of the electronic cigarette, and permanently disabling the electronic cigarette.

It should be noted that, the above preset value may be set according to components of the e-liquid, a capacity of the e-liquid inside the electronic cigarette and a statistic value of a usage amount of the e-liquid in each puff, and may be set as 180 puffs, 190 puffs or the like, which is not limited herein.

Optionally, the smoking trigger module 5 may be the airflow sensor and may be installed in an airflow channel of the electronic cigarette.

Optionally, the control module 2 may further include an anti-interference sub-module 23. The anti-interference sub-module 23 is configured to record a trigger hold time of the airflow sensor, and transmit a signal indicating that a present record is invalid to the main control sub-module 22 in the case that the trigger hold time is less than a preset value. It can be understood that, the anti-interference sub-module 23 may be implemented by a timing circuit of a microprocessor, which is not limited herein.

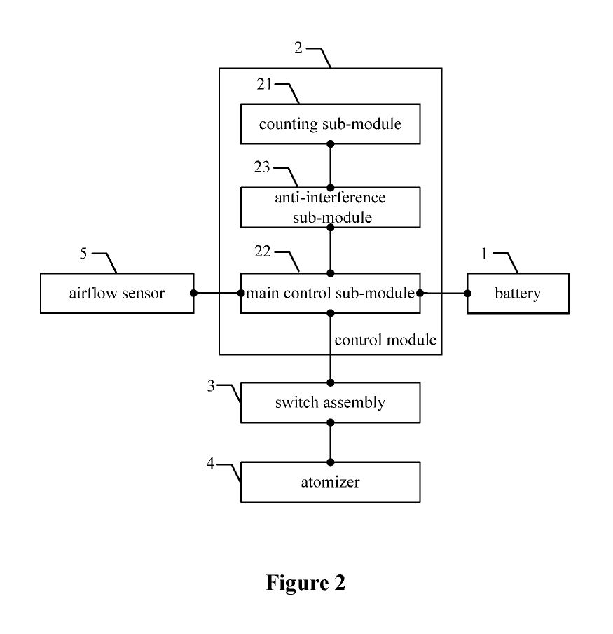

Reference is made to FIG. 2 which shows a second embodiment of the electronic cigarette with a limited service life according to the present application. Unlike the first embodiment, in this embodiment, the smoking trigger module 5 is embodied as the airflow sensor and an anti-interference sub-module 23 is further provided. A smoking time duration may affect a usage amount of the e-liquid in the electronic cigarette, and in the case that a smoking time duration is short, the usage amount of the e-liquid is small, thus, if a smoking action with a short smoking time duration is considered as one puff and is counted in the number of puffs, the following situation may happen, that the capacity of the e-liquid is still in a range capable of ensuring a good taste of the electronic cigarette when the electronic cigarette is turned off or gives a warning. The anti-interference sub-module 23 may record a trigger hold time of the airflow sensor, and transmit a signal indicating that the present record is invalid to the main control sub-module 22 in the case that the trigger hold time is less than a preset value, thereby avoiding the above situation. And the above preset value may be set according to a specific structure of the electronic cigarette and components of the e-liquid, which is not limited herein.

Optionally, the smoking trigger module 5 is a button input module.

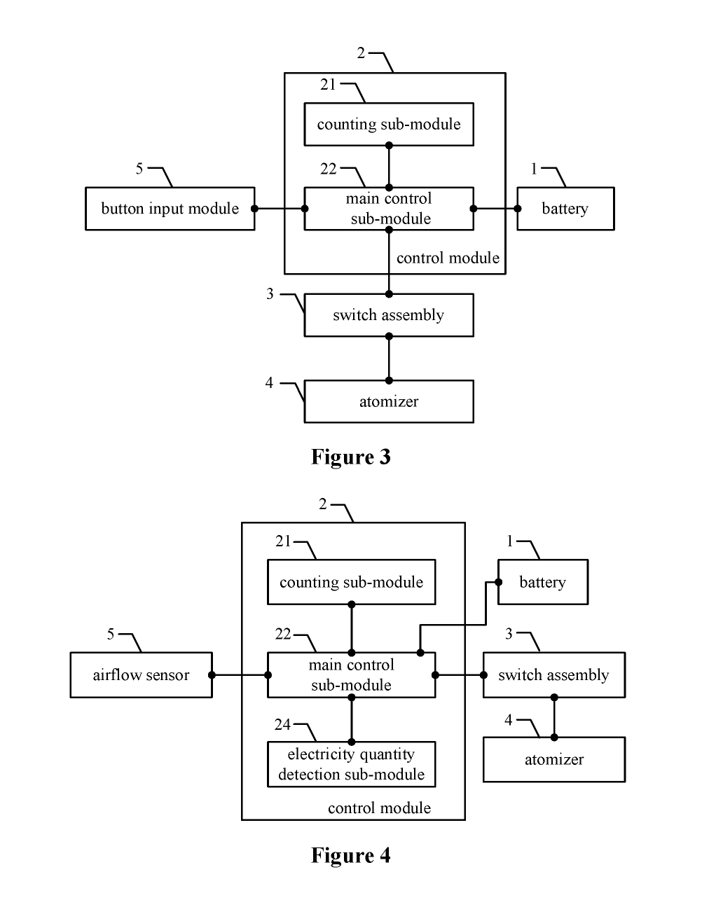

Reference is made to FIG. 3 which is a third embodiment of the electronic cigarette with a limited service life according to the present application. Unlike the first embodiment, in this embodiment, the smoking trigger module 5 is embodied as the button input module. Through the button input module provided on the electronic cigarette, when smoking, a user presses a button in the button input module to trigger the control module 2, and controls, by the control module 2, the battery 1 to supply power to the atomizer 4. In this way, various smoking requirements of different users can be met.

Optionally, the control module 2 may further include an electricity quantity detection sub-module 24. The electricity quantity detection sub-module 24 is connected to the main control sub-module 22 and is configured to detect an electricity quantity of the battery.

Reference is made to FIG. 4 which is a fourth embodiment of the electronic cigarette with a limited service life according to the present application. Unlike the first embodiment, in this embodiment, an electricity quantity detection sub-module 24 is further provided in the electronic cigarette. When the electricity quantity is lower than a normal level, the atomizer 4 in the electronic cigarette cannot reach a normal working temperature due to the insufficient electricity quantity, thereby affecting the taste of the vapor. The provided electricity quantity detection sub-module can transmit a signal indicating a low electricity quantity to the main control sub-module 22 when the electricity quantity is insufficient, and the main control sub-module 22 may turn off the electronic cigarette in response to the signal.

Optionally, the electronic cigarette may further include a warning module 6. The warning module 6 is connected to the main control sub-module 22, and is configured to give a warning in response to a control signal of the main control sub-module 22 in the case that the number counted by the counting sub-module 21 is about to reach or reaches a preset value.

Optionally, the warning module 6 may be embodied as an LED lamp array, a digital tube or a display screen arranged on the electronic cigarette.

Optionally, the warning module 6 may be embodied as a buzzer or a speaker and configured to give a voice warning in response to the control signal of the main control sub-module 22.

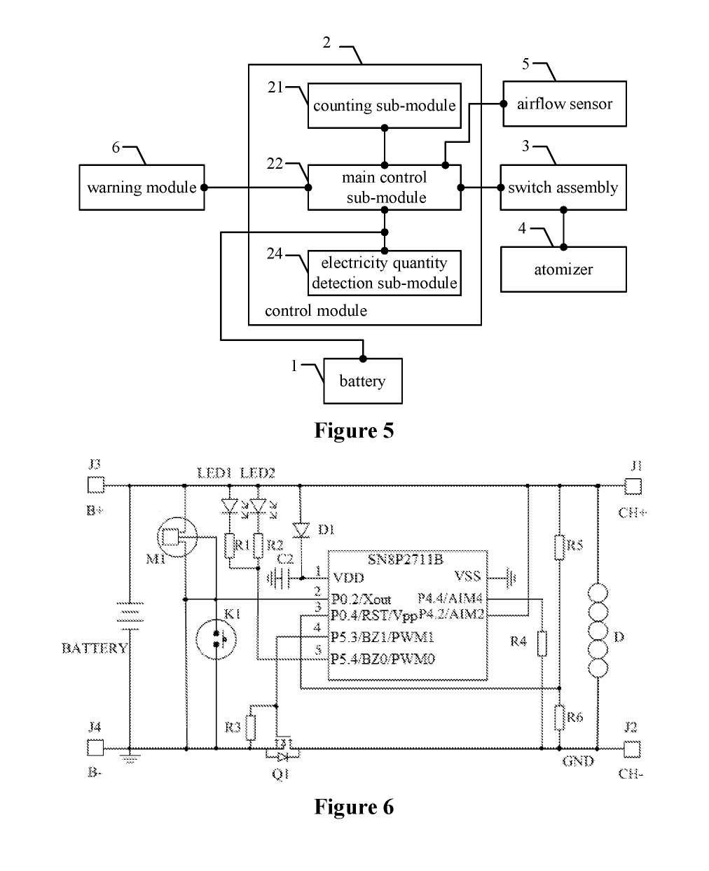

Reference is made to FIG. 5 which is a fifth embodiment of the electronic cigarette with a limited service life according to the present application. Unlike the first embodiment, in this embodiment, a warning module 6 is further provided on the electronic cigarette. Some users may hope to keep smoking instead of forcibly turning off the electronic cigarette when the e-liquid in the electronic cigarette is insufficient, that is, the number of puffs is about to reach the preset vale or reaches the preset value or the electricity quantity is low. In this case, the provided warning module 6 can further improve the humanization degree of the electronic cigarette according to the present application. It should be noted that, the warning function of the warning module 6 may be switched off or switched on by a switch arranged on the electronic cigarette, which is not limited herein.

The warning module 6 may include a display module and/or a voice module. The display module may include an LED lamp array, a digital tube or a display screen. The voice module may include a buzzer or a speaker. The warning information can be presented to the user directly by the display module and/or the voice module.

It should be noted that, the switch assembly 3 has multiple types, such as a transistor switch, a small relay switch or a button switch. The transistor switch may be a field effect tube, a triode or the like. In the case that the switch assembly 3 is embodied as a transistor switch, the transistor switch is electrically connected to the battery, to provide a current required by a heating wire in the atomizer to atomize the e-liquid. Whether the switch assembly 3 needs to be connected to the battery 1 may be designed according to an actual structure, and is not limited herein.

Reference is made to FIG. 6 which is a schematic circuit diagram of an embodiment of the electronic cigarette with a limited service life according to the present application. In the figure, a model number of a control chip in the control module is SN8P2711B. A working process of the circuit is described in detail hereinafter. In smoking, a smoking signal is transferred to the control chip by an airflow sensor M1 or by pressing a button K1. The control chip controls a field effect tube Q1 of the switch assembly to be switched on, to allow the battery BATTERY to supply power to a heating wire D of the atomizer and to allow the heating wire D to atomize the e-liquid, and at the same time, the control chip controls switch indicator lamps LED1 and LED2 to be switched on. In the figure, a capacitor C2 is configured to supply power to the control chip in the case that a short circuit occurs between CH+ and CH−, to allow the control chip to have a buffering time for switching off the field effect tube Q1, thereby avoiding a danger situation.

The electronic cigarette with a limited service life according to the present application includes the battery 1, the control module 2, the switch assembly 3, the atomizer 4 and the smoking trigger module 5. The control module 2 includes a counting sub-module 21 and the main control sub-module 22. The counting sub-module 21 is connected to the main control sub-module 22, and is configured to record the number of puffs according to the monitoring signal transmitted from the smoking trigger module 5 to the main control sub-module 22. The main control sub-module 22 is connected to the battery 1, the switch assembly 3, the atomizer 4 and the smoking trigger module 5, and is configured to control the electronic cigarette according to the number of puffs and the monitoring signal. With the counter for counting the number of puffs and the detector for detecting the electricity quantity of the battery provided in the electronic cigarette with a limited service life according to the present application, the operation of the electronic cigarette can be directly stopped or stopped after giving a warning, to permanently disable the electronic cigarette, in the case that the number of puffs is about to reach or reaches a preset value or in the case of a low electricity quantity. In this case, a product consistency can be ensured, thereby bringing a good smoking experience to the user. Moreover, with the electricity quantity detection sub-module and the warning module, the practicability and the humanization degree of the electronic cigarette according to the present application can be further improved.

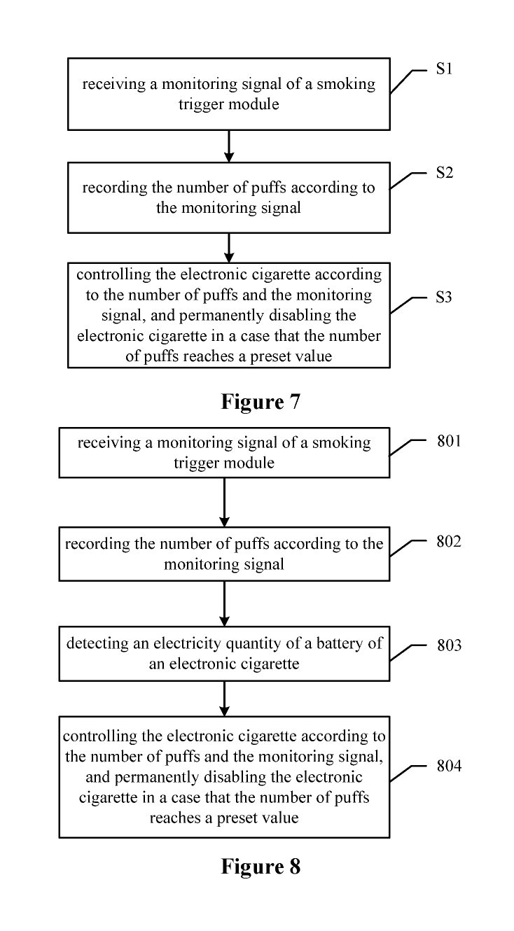

The first embodiment to the fifth embodiment of the electronic cigarette with a limited service life according to the present application are described hereinabove. A first embodiment of a method for limiting a service life of an electronic cigarette according to the present application is described hereinafter. Referring to FIG. 7, the first embodiment of the method for limiting the service life of the electronic cigarette according to the present application includes the following steps S1 to S3.

Step S1 may include receiving a monitoring signal of a smoking trigger module.

Specifically, in the step S1, the monitoring signal of the smoking trigger module is received by a main control sub-module. The smoking trigger module keeps detecting whether a user is smoking the electronic cigarette, and transmits a monitoring signal to the control module in the case that it is detected that the user is smoking the electronic cigarette.

Step S2 may include recording the number of puffs according to the monitoring signal.

Specifically, in the step S2, the counting sub-module records the number of puffs according to the monitoring signal. After receiving the monitoring signal, the control module may record the number of puffs according to the monitoring signal. Specifically, for example, the number of puffs pre-stored in a counting unit in the control module may be zero, and the counting unit adds 1 to the number of puffs each time when a monitoring signal is received. Or, the number of puffs pre-stored in the counting unit in the control module may be a specified value, such as 180 puffs or 190 puffs; and in this case, the counting unit subtracts 1 from the number of puffs each time when a monitoring signal is received.

Step S3 may include controlling the electronic cigarette according to the number of puffs and the monitoring signal, and permanently disabling the electronic cigarette when the number of puffs reaches a preset value.

Specifically, in the step S3, the main control sub-module controls the electronic cigarette according to the number of puffs and the monitoring signal. When the number of puffs reaches a preset value of the number of puffs, the control module may control the battery to stop supplying power to an atomizer according to the number of puffs, to permanently disable the electronic cigarette.

A second embodiment of a method for limiting a service life of the electronic cigarette according to the present application is further described hereinafter. Referring to FIG. 8, the second embodiment of the method for limiting the service life of the electronic cigarette according to the present application includes the following steps 801 to 804.

Step 801 may include receiving a monitoring signal of a smoking trigger module.

The smoking trigger module keeps detecting whether a user is smoking the electronic cigarette, and transmits a monitoring signal to a control module when it is detected that the user is smoking the electronic cigarette.

It should be noted that, in order to improve a rationality of recording the number of puffs, when it is detected that the user is smoking the electronic cigarette, a time period the user smokes the electronic cigarette may also be recorded, and in the case that the time period during which the user smokes the electronic cigarette is in a preset reasonable range, the monitoring signal is labeled as a reasonable signal and is transmitted to the control module.

Step 802 may include recording the number of puffs according to the monitoring signal.

After receiving the monitoring signal, the control module may record the number of puffs according to the monitoring signal. Specifically, for example, the number of puffs pre-stored in a counting unit in the control module may be zero, and the counting unit adds 1 to the number of puffs each time when a monitoring signal is received. Or, the number of puffs pre-stored in the counting unit in the control module may be a specified value, such as 180 puffs or 190 puffs; and in this case, the counting unit subtracts 1 from the number of puffs each time when a monitoring signal is received.

Step 803 may include detecting an electricity quantity of a battery of the electronic cigarette.

An atomizer inside the electronic cigarette may fail to reach a normal working temperature due to an insufficient electricity quantity, and a taste of the vapor may be affected consequently, thus the electricity quantity of the battery of the electronic cigarette may be detected and transmitted to the control module.

It should be noted that, in the embodiments of the present application, the step 803 may be performed before or after any previous steps of the step 804, instead of being limited to be performed after the step 802 and before the step 804, which is not limited herein.

Step 804 may include controlling the electronic cigarette according to the number of puffs and the monitoring signal, and permanently disabling the electronic cigarette in the case that the number of puffs reaches a preset value.

In the case that the number of puffs reaches a specified value of the number of puffs or it is detected that the electricity quantity of the battery is insufficient, the control module may control the electronic cigarette according to the number of puffs, the electricity quantity and a hardware condition of the electronic cigarette. Specifically, the control module may control the electronic cigarette to stop working to permanently disable the electronic cigarette, or may give a warning and then control the electronic cigarette to stop working to permanently disable the electronic cigarette, thereby making the user to use the electronic cigarette conveniently.

By recording the number of puffs of the user, the method for limiting the service life of the electronic cigarette according to the present application may directly stop the operation of the electronic cigarette or stop the operation of the electronic cigarette after giving a warning in the case that the number of puffs is about to reach or reaches a preset value or the electricity quantity is low, to permanently disable the electronic cigarette, thereby ensuring a product consistency and providing a good smoking experience to the user. Moreover, with the step of detecting the electricity quantity, the practicability and humanization degree of the electronic cigarette according to the present application can be further improved. After the operation of the electronic cigarette according to the present application is stopped, the electronic cigarette can no longer atomize the e-liquid, to permanently disable the electronic cigarette.

The electronic cigarette with a limited service life and the method for limiting the service life of the electronic cigarette according to the present application are described in detail hereinbefore. The principle and the embodiments of the present application are illustrated herein by specific examples. The above description of examples is only intended to help the understanding of the method and concept of the present application. It should be noted that, for the person skilled in the art, a few of modifications and improvements may be made to the present application without departing from the principle of the present application, and these modifications and improvements are also deemed to fall into the scope of the present application defined by the claims.

What is claimed is:

1. An electronic cigarette with a limited service life, comprising a battery, a switch assembly and an atomizer, and further comprising a control module and a smoking trigger module; wherein,
the control module comprises a counting sub-module and a main control sub-module;
the counting sub-module is connected to the main control sub-module, and is configured to record the number of puffs according to a monitoring signal transmitted from the smoking trigger module to the main control sub-module; and
the main control sub-module is connected to the battery, the switch assembly and the smoking trigger module, and is configured to control the electronic cigarette according to the number of puffs and the monitoring signal, and permanently disable the electronic cigarette in a case that the number of puffs reaches a preset value.

2. The electronic cigarette with a limited service life according to claim 1, further comprising a warning module; wherein the warning module is connected to the main control sub-module, and is configured to give a warning in response to a control signal of the main control sub-module in a case that the number of puffs counted by the counting sub-module is about to reach or reaches the preset value.

3. The electronic cigarette with a limited service life according to claim 1, wherein the switch assembly is a transistor switch, and the transistor switch is electronically connected to the battery, the main control sub-module and the atomizer.

4. The electronic cigarette with a limited service life according to claim 1, wherein the smoking trigger module comprises an airflow sensor and/or a button input module.

5. The electronic cigarette with a limited service life according to claim 4, wherein the smoking trigger module is the airflow sensor and is installed inside an airflow channel of the electronic cigarette.

6. The electronic cigarette with a limited service life according to claim 5, wherein the control module further comprises an anti-interference sub-module; and the anti-interference sub-module is configured to record a trigger hold time of the airflow sensor, and transmit a signal indicating that a present record is invalid to the main control sub-module in a case that the trigger hold time is less than a preset value.

7. The electronic cigarette with a limited service life according to claim 1, wherein the control module further comprises an electricity quantity detection sub-module; and the electricity quantity detection sub-module is connected to the main control sub-module and is configured to detect an electricity quantity of the battery.

8. The electronic cigarette with a limited service life according to claim 2, wherein the control module further comprises an electricity quantity detection sub-module; and the electricity quantity detection sub-module is connected to the main control sub-module and is configured to detect an electricity quantity of the battery.

9. The electronic cigarette with a limited service life according to claim 3, wherein the control module further comprises an electricity quantity detection sub-module; and the electricity quantity detection sub-module is connected to the main control sub-module and is configured to detect an electricity quantity of the battery.

10. The electronic cigarette with a limited service life according to claim 4, wherein the control module further comprises an electricity quantity detection sub-module; and the electricity quantity detection sub-module is connected to the main control sub-module and is configured to detect an electricity quantity of the battery.

11. The electronic cigarette with a limited service life according to claim 5, wherein the control module further comprises an electricity quantity detection sub-module; and the electricity quantity detection sub-module is connected to the main control sub-module and is configured to detect an electricity quantity of the battery.

12. The electronic cigarette with a limited service life according to claim 6, wherein the control module further comprises an electricity quantity detection sub-module; and the electricity quantity detection sub-module is connected to the main control sub-module and is configured to detect an electricity quantity of the battery.

13. A method for limiting a serving life of an electronic cigarette, the electronic cigarette comprising a battery, a switch assembly, an atomizer, a control module and a smoking trigger module; the control module comprising a counting sub-module and a main control sub-module; the counting sub-module being connected to the main control sub-module, and the main control sub-module being connected to the battery, the switch assembly and the smoking trigger module, wherein the method comprises:
S1, receiving a monitoring signal of the smoking trigger module;
S2, recording the number of puffs according to the monitoring signal; and
S3, controlling the electronic cigarette according to the number of puffs and the monitoring signal, and permanently disabling the electronic cigarette in a case that the number of puffs reaches a preset value.

14. The method according to claim 13, wherein before the step S3, the method further comprises:
detecting an electricity quantity of the battery of the electronic cigarette.

15. The method according to claim 13, wherein,
the step S1 further comprises receiving, by the main control sub-module, the monitoring signal of the smoking trigger module;
the step S2 further comprises recording, by the counting sub-module, the number of puffs according to the monitoring signal; and
the step S3 further comprises controlling, by the main control sub-module, the electronic cigarette according to the number of puffs and the monitoring signal, and controlling the battery to stop supplying power to the atomizer in the case that the number of puffs reaches the preset value, to permanently disable the electronic cigarette.
